(19) United States
(12) Patent Application Publication
(10) Pub. No.: US 2012/0141546 A1
(43) Pub. Date: Jun. 7, 2012

(54) OIL-BASED PREPARATION

(75) Inventors: Venkata-Rangarao Kanikanti, Leverkusen (DE); Achim Harder, Koeln (DE); Thomas Bach, Wuppertal (DE)

(73) Assignee: BAYER ANIMAL HEALTH GMBH, Leverkusen (DE)

(21) Appl. No.: 13/202,739

(22) PCT Filed: Mar. 5, 2010
(86) PCT No.: PCT/EP2010/001396

§ 371 Date: Aug. 22, 2011

(30) Foreign Application Priority Data
Mar. 10, 2009 (DE) 10200902423.3

(51) Int. Cl. A61K 38/15; A61P 33/02; A61P 33/10; A61K 9/10

(52) U.S. Cl. 424/400; 514/4.6

(57) ABSTRACT

The present invention relates to an oil-based preparation comprising a triazine which is active against parasitic protozoans and an anthelmintic cyclodepsipeptide, which preparation is particularly suitable for the oral application of the active substance combination in animals.

The present invention relates to an oil-based preparation comprising a triazine which is active against parasitic protozoans and an anthelmintic cyclodepsipeptide, which preparation is suitable in particular for oral application of the active substance combination in animals.

Triazines, in particular toltrazuril and ponzazuril, and their activity against parasitic protozoans such as, for example, coccidia, are known from a series of publications, see, inter alia, DE-OS 27 18 799, DE-OS 24 137 22, EP-A 116 175, EP-A 1 246 624, EP-A 1140 102, EP-A 1 311 491 and WO 2008/145281. WO 99/62519 discloses semi-solid aqueous preparations of toltrazuril-sulphone (ponazuril).

Cyclic depsipeptides and their endoparasiticidal activity are known: enniatins and other 18-membered cyclic depsipeptides (EP-A 644 883, EP-A 658 551, EP-A 669 343, WO 95/27498); 24-membered cyclic depsipeptides (EP-A 626 376, EP-A 626 375, EP 787 141, EP-A 903 347, EP-A 973 756, WO 98/55469, WO 99/47506, WO 00/14079, WO 98/37088, WO 99/67281), cyclic depsipeptides with 12 ring atoms (EP-A 664 297). Cyclic octadepsipeptides such as PH 022 and emodepside and their activity against endoparasites (for example against intestinal nematodes and tissue-dwelling nematodes) are likewise already known, see, for example, EP-A 382 173, EP-A 634 408. The following may further be mentioned in connection with depsipeptides, in particular emodepside: EP 662 326, EP-A 1 259 250 and WO05/055973.

There was therefore a need for pharmaceutical preparations which are suitable in particular for oral application and which comprise triazines which are active against parasitic protozoans and anthelmintic depsipeptides (see, for example, Barutzki D; Schaper R “Endoparasites in dogs and cats in Germany 1999-2002”. Parasitol. Res. 2003 July; 90 Suppl. 3:p. 148-50. Epub 2003, Aug. 19). The triazines must be available systemically, i.e. in particular in the case of oral administration. They must pass from the gastrointestinal tract into the bloodstream, where they act against the parasitic protozoans. In contrast, the anthelmintic depsipeptides should become active in the intestine since this is where it is intended to control the worms locally. It is not necessary for the anthelmintic depsipeptides to pass into the bloodstream and rather it is undesirable, in order to avoid any side effects.

The object was therefore to find a formulation which makes the depsipeptide locally available in the intestine and the triazine in the bloodstream. What is important in this context is that each of the two active substances are available in sufficient amounts at the correct site of activity. The depsipeptide should only reach the bloodstream in amounts which are not critical for the treated animal with regard to side effects.

The invention relates to: preparations comprising a triazine which is active against parasitic protozoans and has a particle size d(90)≦15 μm and an anthelmintic cyclodepsipeptide in an oil base.

Triazines which are active against parasitic protozoans are in particular those of the formulae (I) or (II)

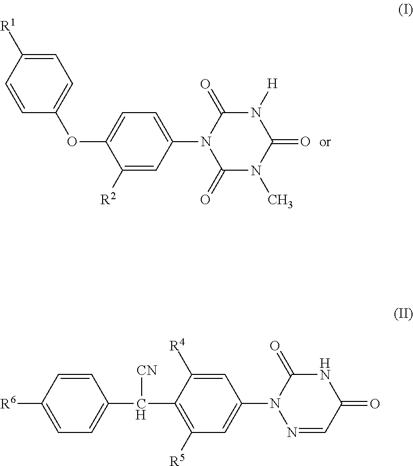

in which
R¹ represents R³—SO₂— or R³—S—,
R² represents alkyl, alkoxy, halogen or SO₂N(CH₃)₂ and
R³ represents haloalkyl,
R⁴ and R⁵ independently of one another represent hydrogen or Cl and
R⁶ represents fluorine or chlorine, and their physiologically acceptable salts.

The triazines are well known per se as active substances in particular against coccidial infections, examples which may be mentioned are the triazinetriones such as toltrazuril and ponazuril and the triazinediones such as clazuril, diclazuril and letrazuril.

The triazinediones are represented by the formula (II):

clazuril (R⁴═Cl, R⁵═H, R⁶=Cl in formula (II))
letrazuril (R⁴═Cl, R⁵═Cl, R⁶═F in formula (II)) and
diclazuril (R⁴═Cl, R⁵═Cl, R⁶═Cl in formula (II)).

Among these 1,2,4-triazinediones, diclazuril is most preferred.

Especially preferred in accordance with the invention are the triazinetriones of the formula (I) as active substances:
R² preferably represents alkyl or alkoxy having in each case up to 4 carbon atoms, especially preferably methyl, ethyl, n-propyl, i-propyl.
R³ preferably represents perfluoroalkyl having 1 to 3 carbon atoms, especially preferably trifluoromethyl or pentafluoroethyl.

The preferred triazinetriones are represented by the formula (I):

toltrazuril (R¹═R³—S—, R²═CH₃, R³═CF₃)
ponazuril (R¹=R³—SO₂—. R²═CH₃, R³═CF₃)

Depsipeptides are similar to the peptides and differ from the latter by the fact that one or more α-amino acid units are replaced by α-hydroxycarboxylic acid units. Those which are preferably employed in accordance with the invention are cyclic depsipeptides, in particular those having 24 ring atoms (cyclooctadepsipeptides).

Among the cyclic depsipeptides having 24 ring atoms, the compound PF 1022 of the formula (IIIa) hereinbelow, which is known from EP-OS 382 173, may be mentioned:

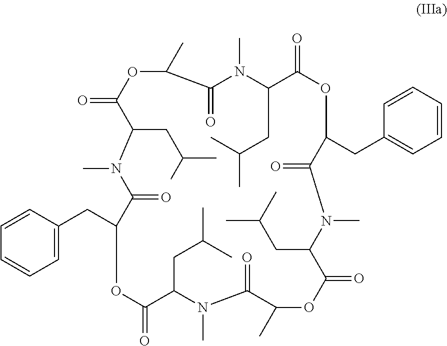

Moreover, depsipeptides which may be mentioned are the compounds known from the PCT application WO 93/19053.

From WO 93/19053, the compounds of the formula (IIIb) hereinbelow may be mentioned in particular:

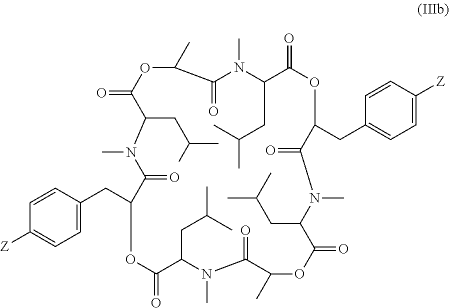

in which
Z represents N-morpholinyl, amino, mono- or dimethylamino.

Moreover, compounds of the formula (IIIc) hereinbelow may be mentioned:

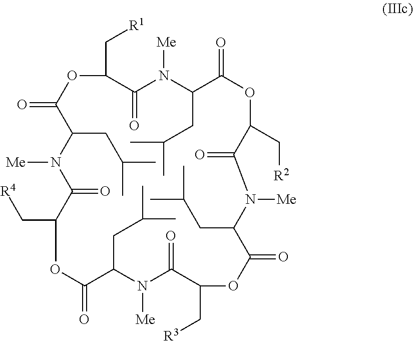

in which
R¹, R², R³, R⁴ independently of one another represent hydrogen, C₁-C₁₀-alkyl or aryl, in particular phenyl, which are optionally substituted by hydroxyl, C₁-C₁₀-alkoxy or halogen.

The cyclic depsipeptides having 24 ring atoms also include compounds of the general formula (IIId)

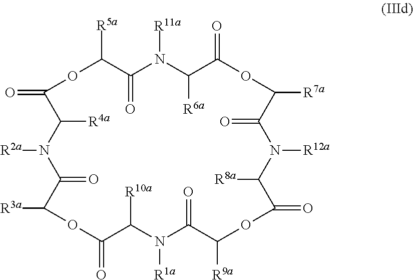

in which
R^{1a}, R^{2a}, R^{11a} and R^{12a} independently of one another represent C₁₋₈-alkyl, C₁₋₈-haloalkyl, C₃₋₄-cycloalkyl, aralkyl, aryl,
R^{3a}, R^{5a}, R^{7a}, R^{9a} independently of one another represent hydrogen or straight-chain or branched C₁₋₈-alkyl which can optionally be substituted by hydroxyl, C₁₋₄-alkoxy, carboxyl,

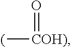

carboxamide,

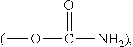

imidazolyl, indolyl, guanidino, —SH or C₁₋₄-alkylthio, and represent furthermore aryl or aralkyl, each of which can be substituted by halogen, hydroxyl, C₁₋₄-alkyl, C₁₋₄-alkoxy,
R^{4a}, R^{6a}, R^{8a}, R^{10a} independently of one another represent hydrogen, straight-chain C₁₋₅-alkyl, C₂₋₆-alkenyl, C₃₋₇-cycloalkyl, each of which can optionally be substituted by hydroxyl, C₁₋₄-alkoxy, carboxyl, carboxamide, imidazolyl, indolyl, guanidino, SH or C₁₋₄-alkylthio, and represent aryl or aralkyl, each of which can be substituted by halogen, hydroxyl, C₁₋₄-alkyl, C₁₋₄-alkoxy, and their optical isomers and racemates.

It, is preferred to employ compounds of the formula (IIId) in which

R^{1a}, R^{2a}, R^{11a} and R^{12a} independently of one another represent methyl, ethyl, propyl, isopropyl, n-, s-, t-butyl or phenyl, which is optionally substituted by halogen, C₁₋₄-alkyl, OH, C₁₋₄-alkoxy, and represent benzyl or phenylethyl, each of which can optionally be substituted by the radicals given for phenyl;
R^{3a} to R^{10a} have the abovementioned meanings.

Especially preferred are compounds of the formula (IIId) in which

R^{1a}, R^{2a}, R^{11a} and R^{12a} independently of one another represent methyl, ethyl, propyl, isopropyl or n-, s-, t-butyl,
R^{3a}, R^{5a}, R^{7a}, R^{9a} represent hydrogen; straight-chain or branched C₁₋₈-alkyl, in particular methyl, ethyl, propyl, i-propyl, n-, s-, t-butyl, each of which can optionally be substituted by C₁₋₄-alkoxy, in particular methoxy, ethoxy, imidazolyl, indolyl or C₁₋₄-alkylthio, in particular methylthio, ethylthio, and furthermore represent phenyl, benzyl or phenethyl, each of which can optionally be substituted by halogen, in particular chlorine.
R^{4a}, R^{6a}, R^{8a}, R^{10a} independently of one another represent hydrogen; methyl, ethyl, n-propyl, n-butyl, vinyl, cyclohexyl, each of which can optionally be substituted by methoxy, ethoxy, imidazolyl, indolyl, methylthio, ethylthio, and represent isopropyl, s-butyl, and furthermore represent optionally halogen-substituted phenyl, benzyl or phenylethyl.

The abovementioned cyclooctadepsipeptides are known and can be prepared by known processes. Thus, they can be obtained, for example, by the processes described in EP-A-382 173, DE-A 4 317 432, DE-A 4 317 457, DE-A 4 317 458, EP-A-634 408, EP-A-718 293, EP-A-872 481, EP-A-685 469, EP-A-626 375, EP-A-664 297, EP-A-669 343, EP-A-787 141, EP-A-865 498, EP-A-903 347.

Depsipeptides which are very especially preferred in accordance with the invention are PF 1022 A (see formula (IIIa) and emodepside (PF 1022-221, compound of the formula (IIIb) in which the two radicals Z represent the morpholinyl radical). The INN emodepside represents the compound with the systematic name: cyclo[(R)-lactoyl-N-methyl-L-leucyl-(R)-3-(p-morpholinophenyl)lactoyl-N-methyl-L-leucyl-(R)-lactoyl-N-methyl-L-leucyl-(R)-3-(p-morpholinophenyl)lactoyl-N-methyl-L-leucyl.

Depending on their structure, the active substances may be present in stereoisomeric forms or as stereoisomer mixtures, for example as enantiomers or racemates or diastereomers or diastereomer mixtures. Not only the stereoisomer mixtures, but also the pure stereoisomers may be used in accordance with the invention; for example, the enantiomers or diastereomers or their respective mixtures may be employed.

If the active substances can exist in tautomeric forms, the present invention also comprises the use of the tautomeric forms.

The following may furthermore be used: salts of the active substances with pharmaceutically acceptable acids or bases, and also solvates, in particular hydrates, of the active substances or of their salts.

If appropriate, the active substances may also be employed in the form of their salts, solvates and solvates of the salts.

Salts which are preferred within the scope of the present invention are physiologically acceptable salts of the active substances.

Physiologically acceptable salts of the active substances comprise, depending on the structure of the active substance, acid addition salts of mineral acids, carboxylic acids and sulfonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

If appropriate, physiologically acceptable salts of the active substances also comprise salts of conventional bases such as, by way of example and by preference, alkali metal salts (for example sodium and potassium salts), alkaline earth metal salts (for example calcium and magnesium salts) and ammonium salts derived from ammonia or organic amines having 1 to 16 C atoms, such as by way of example and by preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Within the scope of the invention, solvates refers to those forms of the active substances which, in the solid or liquid state, form a complex by coordination with solvent molecules. Hydrates are a specific form of the solvates where the coordination is with water.

Moreover, the present invention also relates to prodrugs of the active substances. The term “prodrugs” comprises compounds which themselves may be biologically active or inactive, but which are reacted (for example metabolically or hydrolytically) during their residence time in the body to give the actual active substance.

If appropriate, the products according to the invention may comprise further active substances. Preferred examples which may be mentioned are praziquantel or macrocyclic lactones (for example ivermectin, moxidectin and others).

The active substances are present in the preparations according to the invention in an oil base. Since, as a rule, they are sparingly soluble in the oil base, they are usually suspended therein. Suitable oil bases are preferably liquid pharmaceutically acceptable oily substances. Preferably, these are pharmaceutically acceptable oily fatty acid triglycerides, in particular those with aliphatic fatty acids comprising 8 to 20 carbon atoms. Especially preferred examples are natural vegetable oils such as, for example, sunflower oil, soya oil, castor oil, sesame seed oil, almond oil, rapeseed oil, wheat germ oil or fish oil, or else medium-chain triglycerides (MTCs) such as, for example, caprylic/capric triglyceride (Miglyol®812). Others which are suitable as the oil base are the natural vegetable oils apricot kernel oil, canola oil or modified vegetable oils such as maleated soybean oil. Sunflower oil is especially preferred. The substances which are suitable as the oil base may also be employed as mixtures. Usually, the preparations comprise the oil base in amounts of from 10 to 99% by weight, preferably from 50 to 98% by weight, especially preferably from 80 to 95% by weight.

The triazines are employed in micronized form. The micronization may be carried out using customary methods, such as, for example, grinding in a bead mill, which, in the present case, can advantageously already be carried out in the suitable oily medium. Here, the dispersed triazine has a particle size (measured by laser diffraction, Malvern Mastersizer® 2000) of d(90)≦15 μm, preferably d(90)≦12 μm, especially preferably d(90)≦10 μm, and very especially preferably d(90) equal to 9 μm or less. For the purposes of the present invention, d(90) is understood as meaning a volume-related particle size distribution where 90% of all particles have dimension (diameter) of this value or less. The particle sizes specified here were determined by the laser-diffraction method using a Mastersizer 2000 apparatus (dispersing unit Hydro 2000G) from Malvern and the evaluation mode of Fraunholer diffraction since the refraction indices of the active substances are not known. Here, a suitable sample quantity is predispersed, with stirring, using 2-3 ml of a dispersion medium (low-viscosity mineral oil). Then, the dispersion is placed into the dispersing unit of the apparatus and measured. The evaluation software shows the particle size as d(90) values etc.

Preferably, the preparations according to the invention furthermore comprise a thickener such as, for example, colloidal silica or solid fatty bases, in particular glycerol esters. Preferably, these take the form of esters with C12-C24 fatty acids. Glycerol esters which may be mentioned are glycerol diesters such as, for example, glycerol dibehenate (Compritol 888 ATO); glycerol triesters such as, for example, glycerol esters of saturated C12-C18 fatty acids (for example Gelucire® 43/01), glycerol trilaurate, glycerol trimyristate, glycerol tripalmitate or glycerol tristearate (for example Dynasan 118), mixtures of glycerol mono-, di- and triesters such as, for example, glycerol palmitostearate (Precirol ATO 5). Others which may be mentioned are triglycerides based on coconut fat, palm oil and/or palm kernel oil (such as, for example, the hard fats commercially available as Witocan®). Mono- or diglycerides of citric and/or lactic acid may also be employed. An example which may be mentioned is glyceryl stearate citrate (Imwitor 372P). Others which may be mentioned are: glyceryl monoesters such as, for example, glyceryl monostearate (Imwitor 491); linoleoyl macrogolglycerides (for example Labrafil M2125 CS); hardened triglycerides based on coconut or palm kernel oil, such as, for example, hydrogenated coco-glycerides (for example Witocan 42/44); hydrogenated castor oil (for example Cutina HR PH). Substances which are preferably employed are glycerol diesters with C12-C24 fatty acids, with glyceryl dibehenate being mentioned as an especially preferred example. The thickener is usually employed in amounts of from 1 to 15% by weight, preferably from 1 to 10% by weight, especially preferably from 2 to 8% by weight. If a liquid suspension is to be prepared, no more than 5% by weight of thickener will, as a rule, be added.

Another possibility is the preparation of pastes which, as a rule, will comprise more than 5% by weight of thickener. In this context, a paste is understood as meaning those preparations which do not flow as the result of their intrinsic weight (as is the case with, for example, toothpaste).

It has been found that preparations with very good stability to sedimentation and degradation are obtained, in particular when using the abovementioned preferred and especially preferred thickeners.

Furthermore, the preparations according to the invention preferably comprise an antioxidant. Antioxidants are, for example, butylhydroxytoluene, butylhydroxyanisole, propyl gallate or tocopherol, or combinations of these antioxidants. It is preferred to employ butylhydroxyanisole (BHA) or, in particular, butylhydroxytoluene (BHT), or combinations of these antioxidants. They are employed in the amounts conventionally used for the respective antioxidant. As a rule, the concentrations are from 0.01 to 3% by weight, preferably from 0.04 to 0.5% by weight.

Furthermore, the preparations preferably comprise a preservative, for example para-hydroxybenzoic esters (parabens) such as methyl 4-hydroxybenzoate, ethyl 4-hydroxybenzoate or propyl 4-hydroxybenzoate. It is preferred to employ sorbic acid. The preservatives may be employed individually or in combination in an amount sufficient to bring about preservation. They are usually present in concentrations of from 0.01 to 1% by weight, preferably from 0.02 to 0.5% by weight, especially preferably from 0.02 to 0.1% by weight.

Furthermore, the preparations optionally comprise sweeteners such as, for example, saccharin, aspartame or cyclamate. Combinations of the sweeteners are also possible. They are employed in the customary concentrations which, as a rule, are in the range of from 0.05 to 0.5% by weight.

Furthermore, the preparations optionally comprise flavourings or aroma substances. Preferred are meat flavourings such as, for example “Artificial Beef Flavor” (Pharma Chemie, Inc., 1877 Midland Street, Syracuse, Nebr. 48666, in particular the product known as PC-0125) and, preferably, dry liver powder (for example from pigs or chickens). The flavourings or aroma substances are employed in the customary concentrations, which are, as a rule, in the range of from 1 to 20% by weight, preferably from 2 to 10% by weight, especially preferably from 3 to 8% by weight.

The preparations according to the invention can be prepared in a manner known per se by dissolving or dispersing the constituents in the oil base. In this context, it may, if appropriate, be advantageous to warm the mixture. In the event that a solid fatty base is employed, a possible procedure is to warm the oil base to a temperature at which the solid fatty base melts. Then, the fatty base and the other constituents are added, with stirring, and the mixture is cooled with continued stirring.

The products according to the invention have favourable toxicity to warm-blooded species and are suitable for controlling pathogenic endoparasites which occur in humans and in animal keeping and animal breeding in livestock, breeding animals, zoo animals, laboratory animals, test animals and pets. In this context, they are active against all or individual developmental stages of the pests and against resistant and normally-sensitive species. By controlling the pathogenic endoparasites, it is intended to reduce disease, deaths and reduce performance (for example in the production of meat, milk, wool, hides, eggs, honey and the like) so that more economical and simpler animal keeping is possible by employing the active substances. The pathogenic endoparasites include cestodes, trematodes, nematodes, Acantocephala. Examples which may be mentioned are:

From the order Pseudophyllidea, for example: Diphyllobothrium spp., Spirometra spp., Schistocephalus spp., Ligula spp., Bothridium spp., Diphlogonoporus spp.

From the order Cyclophyllidca, for example: Mesocestoides spp., Anoplocephala spp., Paranoplocephala spp., Moniezia spp., Thysanosomsa spp., Thysaniezia spp., Avitellina spp., Stilesia spp., Cittotaenia spp., Anhyra spp, Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

From the subclass Monogenea, for example: Gyrodactylus spp., Dactylogyrus spp., Polystoma spp.

From the subclass Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp-, Cotylophoron spp., Gigantocotyle spp., Fischocderius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

From the order Enoplida, for example: Trichuris spp., Capillaria spp., Trichlomosoides spp., Trichinella spp.

From the order Rhabditia, for example: Micronema spp., Strongyloides spp.

From the order Strongylida, for example: Strongylus spp., Triodontophorus spp., Oesophagodontus spp., Trichonema spp., Gyalocephalus spp., Cylindropharynx spp., Poteriostromum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostomum spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Angiostrongylus spp., Aelurostrongylus spp. Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp., Cylicocyclus spp., Cylicodontophorus spp.

From the order Oxyurida, for example: Oxyuris spp. Enterobius spp., Passalurus spp., Syphacia spp., Aspiculuris spp., Heterakis spp.

From the order Ascaridia, for example: Ascaris spp., Toxascaris spp., Toxocara spp., Parascaris spp., Anisakis spp., Ascaridia spp.

From the order Spirurida, for example: Gnathostoma spp., Physaloptera spp., Thelazia spp., Gongylonema spp., Habronema spp., Parabronema spp., Draschia spp., Dracunculus spp.

From the order Filariida, for example: Stephanofilaria spp., Parafilaria spp., Setaria spp., Loa spp., Dirofilaria spp., Litomosoides spp., Brugia spp., Wuchereria spp., Onchocerca spp.

From the group Gigantohynchida, for example: Filicollis spp., Moniliformis spp., Macracanthorhynchus spp., Prosthenorchis spp.

What is decisive here is, of course, the spectrum of action of the active substance(s) employed. Thus, for example, the depsipeptides will, as a rule, have good activity against trematodes, nematodes, Acanthocephala, but will generally not show any activity which is relevant in practice against cestodes (tapeworms). In contrast, for example praziquantel is essentially only active against cestodes.

Also preferred is the control of nematodes such as, for example, from the order Strongylida, for example: Ancylostoma spp., Uncinaria spp., Angiostrongylus spp.; Aerulostrongylus spp.; from the order Ascaridia, for example: Toxocara spp.; Toxascaris spp.; from the order Filariida, for example: Dirofilaria spp.

Especially preferred is the use of the products according to the invention for controlling

Strongylida, in particular Ancylostoma spp., Uncinaria spp., and for controlling Ascaridia, in particular Toxocara spp., Toxascaris spp.

If the preparations comprise an active substance with activity against tapeworms, it is preferred to control for example Taenia spp.

Parasitic protozoans are in particular coccidia. The coccidia include:

Mastigophora (Flagellata), such as, for example, Trypanosomatidae, for example, Trypanosoma brucei, T. gambiense, T. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example Giardia lamblia, G. canis, Sarcomastigophora (Rhizopoda) such as Entamoebidae, for example Entamoeba histolytica, Hartmanellidae, for example, Acanthamoeba sp., Hartmanella sp., Apicomplexa (Sporozoa) such as Eimeridae, for example Eimeria acervulina, E. adenoides, E. alabahmensis, E. anatis, E. anseris, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenclla, E. truncata, E. truttae, E. zuernii, Globidium spec., Isospora belli, I. canis, I. fells, I. ohioensis, I. rivolta, I. spec., I. suis, Neospora caninum, N. hugesi, Cystisospora spec., Cryptosporidium spec. such as Toxoplasmadidae, for example Toxoplasma gondii, such as Sarcocystidae, for example Sarcocystis bovicanis, S. bovihominis, S. neurona, S. ovicanis, S. ovifelis, S. spec., S. suihominis such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Furthermore Myxospora and Microspora, for example Glugea spec. Nosema spec.

Furthermore Pneumocystis carinii, and also Ciliophora (Ciliata) such as, for example, Balantidium coli, Ichthiophthirius spec., Trichodina spec., Epistylis spec.

Those protozoans which belong to the Apicomplexa, especially Isospora spp., in particular Isospora canis and Isospora fells, must be emphasized in particular.

The livestock include in particular mammals such as, for example, cattle, horses, sheep, pigs, goats, camels, water buffalo, donkeys, rabbits, fallow deer, reindeer, fur bears such as, for example, mink, chinchilla, racoon. Preferred among the mammalian livestock are cattle, sheep and pigs. Also included for the use according to the invention are the following animal species, which are not mammals, but also count as livestock: birds, such as, for example, chickens, geese, turkeys, ducks; freshwater and saltwater fish such as, for example, trout, carps, eels; reptiles; insects such as, for example, honeybee and silkworm.

Other pets preferably include cats and, in particular, dogs. In pets, it is preferred to control the abovementioned parasites in kittens and, in particular, puppies. The puppies are, as a rule, 1 to 6 months, preferably 2 to 14 weeks, of age.

The application may be effected prophylactically or therapeutically.

The preparations are preferably applied orally.

The preparations comprise the active substances in each case in concentrations of 10 ppm to 90% by weight, preferably from 50 ppm to 50% by weight, especially preferably from 100 ppm to 20% by weight, preferably from 100 ppm to 10% by weight.

The preparations according to the invention preferably comprise from 0.01 to 3% by weight, preferably from 0.05 to 1% by weight, especially preferably from 0.1 to 0.2% by weight, of depsipeptide.

Preferably, the preparations according to the invention comprise from 0.1 to 10% by weight, preferably from 1 to 6% by weight, especially preferably from 2 to 4% by weight of triazine.

The weight ratio of depsipeptide to triazine in the products according to the invention depends on a variety of factors, but will, as a rule, be in the range of from 1:99 to 50:50, preferably from 1:99 to 30:70.

The dose rate of the triazine can vary as a function of the animal species. Usual dose rates are from 1 to 60 mg of active substance per kg body weight (mg/kg) of the animal to be treated per day, preferably from 5 to 40 mg/kg and especially preferably from 10 to 30 mg/kg.

Toltrazuril is normally dosed as follows when used for oral application (dose rate per day):

Pig: 15 to 25 mg/kg body weight, in particular approximately 20 mg/kg body weight
Cattle: 10 to 20 mg/kg body weight, in particular approximately 15 mg/kg body weight
Sheep: 15 to 25 mg/kg body weight, in particular approximately 20 mg/kg body weight
Poultry: 10 to 20 mg/kg body weight, in particular approximately 15 mg/kg body weight
Dog: 10 to 20 mg/kg body weight, in particular approximately 15 mg/kg body weight
Cat: 10 to 20 mg/kg body weight, in particular approximately 15 mg/kg body weight

Except for poultry, toltrazuril is only administered once per treatment, so that, for example in pigs, cattle and sheep, the dosage rates stated are both per day and per treatment. In poultry, the dose stated is divided between two consecutive days.

Usual dosage rates of the depsipeptides per day are from 0.1 to 100 mg/kg, preferably from 1 to 50 mg/kg body wcght per day.

For example, emodepside is usually dosed as follows when applied orally (dosage rate per day):

Dog: 1 to 5 mg/kg body weight
Puppies: 0.25 to 2.5 mg/kg body weight
Cattle: 0.5 to 5 mg/kg body weight
Sheep: 0.5 to 5 mg/kg body weight

### EXAMPLES

To prepare the examples, the further constituents are dispersed in the oil base. In the event of a solid fatty base, the oil base is warmed to a temperature at which the solid fatty base melts. Then, the fatty base and the other constituents are added with stirring and the mixture is allowed to cool, while stirring is continued.

In the examples, toltrazuril is employed in micronized form, to be precise at d(90)<15 μm.

### Example 1

| | % w/w |
|---|---|
| Toltrazuril | 2.0 |
| Emodepside | 0.1 |
| Butylhydroxyanisole | 0.1 |
| Colloidal silica (Aerosil 200) | 3.0 |
| Sorbic acid | 0.08 |
| Sunflower oil | to 100.0 |

### Example 2

| | % w/w |
|---|---|
| Toltrazuril | 4.0 |
| Emodepside | 0.1 |
| Butylhydroxyanisole | 0.1 |
| Colloidal silica (Aerosil 200) | 3.0 |
| Sorbic acid | 0.08 |
| Sunflower oil | to 100.0 |

### Example 3

| | % w/w |
|---|---|
| Toltrazuril | 2.0 |
| Emodepside | 0.1 |
| Butylhydroxyanisole | 0.1 |
| Glyceryl dibehenate (Compritol 888ATO) | 3.5 |
| Sorbic acid | 0.08 |
| Sunflower oil | to 100.0 |

### Example 4

| | % w/w |
|---|---|
| Toltrazuril | 4.0 |
| Emodepside | 0.1 |
| Butylhydroxyanisole | 0.1 |
| Glyceryl dibehenate (Compritol 888ATO) | 3.5 |
| Sorbic acid | 0.08 |
| Sunflower oil | to 100.0 |

### Example 5

| | % w/w |
|---|---|
| Toltrazuril | 2.0 |
| Emodepside | 0.1 |
| Butylhydroxyanisole | 0.1 |
| Glyceryl dibehenate (Compritol 888ATO) | 5.0 |
| Sorbic acid | 0.08 |
| Sunflower oil | to 100.0 |

### Example 6

| | % w/w |
|---|---|
| Toltrazuril | 2.0 |
| Emodepside | 0.1 |
| Butylhydroxyanisole | 0.1 |
| Glyceryl dibehenate (Compritol 888ATO) | 7.5 |
| Sorbic acid | 0.08 |
| Sunflower oil | to 100.0 |

### Example 7

| | % w/w |
|---|---|
| Toltrazuril | 2.0 |
| Emodepside | 0.1 |
| Butylhydroxyanisole | 0.1 |
| Glyceryl palmitostearate (Precirol ATO 5) | 3.5 |
| Sorbic acid | 0.08 |
| Sunflower oil | to 100.0 |

### Example 8

| | % w/w |
|---|---|
| Toltrazuril | 2.0 |
| Emodepside | 0.1 |
| Butylhydroxyanisole | 0.1 |
| Glyceryl fatty acid ester (Gelucire 43/01) | 3.5 |
| Sorbic acid | 0.08 |
| Sunflower oil | to 100.0 |

### Example 9

| | % w/w |
|---|---|
| Toltrazuril | 2.0 |
| Emodepside | 0.1 |
| Butylhydroxyanisole | 0.1 |
| Glyceryl dibehenate (Compritol 888ATO) | 3.5 |
| Sorbic acid | 0.08 |
| Miglyol 812 | to 100.0 |

### Example 10

| | % w/w |
|---|---|
| Toltrazuril | 2.0 |
| Emodepside | 0.1 |
| Butylhydroxytoluene | 0.1 |
| Glyceryl dibehenate (Compritol 888ATO) | 3.5 |
| Sorbic acid | 0.08 |
| Sunflower oil | to 100.0 |

### Example 11

| | % w/w |
|---|---|
| Toltrazuril | 2.0 |
| Emodepside | 0.1 |
| Butylhydroxyanisole | 0.1 |
| Glyceryl dibehenate (Compritol 888ATO) | 3.5 |
| Sorbic acid | 0.08 |
| Soya oil | to 100.0 |

### Example 12

| | % w/w |
|---|---|
| Toltrazuril | 2.0 |
| Emodepside | 0.1 |
| Butylhydroxytoluene | 0.1 |
| Glyceryl dibehenate (Compritol 888ATO) | 3.5 |
| Sorbic acid | 0.08 |
| Castor oil | to 100.0 |

### Example 13

| | % w/w |
|---|---|
| Toltrazuril | 2.0 |
| Emodepside | 0.1 |
| Butylhydroxytoluene | 0.1 |
| Glyceryl dibehenate (Compritol 888ATO) | 3.5 |
| Sorbic acid | 0.08 |
| Sesame seed oil | to 100.0 |

### Example 14

| | % w/w |
|---|---|
| Toltrazuril | 2.0 |
| Emodepside | 0.1 |
| Butylhydroxytoluene | 0.1 |
| Glyceryl dibehenate (Compritol 888ATO) | 3.5 |
| Sorbic acid | 0.08 |
| Fish oil | to 100.0 |

### Example 15

| | % w/w |
|---|---|
| Toltrazuril | 2.0 |
| Emodepside | 0.1 |
| Butylhydroxyanisole | 0.04 |
| Glyceryl dibehenate (Compritol 888ATO) | 2.1 |
| Sunflower oil | to 100.0 |

### Example 16

| | % w/w |
|---|---|
| Toltrazuril | 2.0 |
| Emodepside | 0.1 |
| Butylhydroxyanisole | 0.2 |
| Sunflower oil | to 100.0 |

### Example 17

| | % w/w |
|---|---|
| Toltrazuril | 4.0 |
| Emodepside | 0.1 |
| Butylhydroxyanisole | 0.2 |
| Aerosil 200 | 3.2 |
| Sorbic acid | 0.5 |
| Sunflower oil | to 100.0 |

### Example 18

| | % w/w |
|---|---|
| Toltrazuril | 2.0 |
| Emodepside | 0.05 |
| Butylhydroxyanisole | 0.2 |
| Glyceryl dibehenate (Compritol 888ATO) | 3.5 |
| Sorbic acid | 0.3 |
| Caprylic/capric triglyceride (Miglyol 812) | to 100.0 |

### Example 19

| | % w/w |
|---|---|
| Toltrazuril | 2.0 |
| Emodepside | 0.1 |
| Butylhydroxyanisole | 0.2 |
| Glyceryl dibehenate (Compritol 888ATO) | 1.6 |
| Sunflower oil | to 100.0 |

### Example 20

| | % w/w |
|---|---|
| Toltrazuril | 2.0 |
| Emodepside | 0.1 |
| Butylhydroxyanisole | 0.5 |
| Glyceryl dibehenate (Compritol 888ATO) | 3.7 |
| Sorbic acid | 0.5 |
| Sunflower oil | to 100.0 |

### Example 21

| | % w/w |
|---|---|
| Toltrazuril | 4.0 |
| Emodepside | 0.1 |
| Butylhydroxyanisole | 0.5 |
| Glyceryl dibehenate (Compritol 888ATO) | 3.7 |
| Sorbic acid | 0.5 |
| Sunflower oil | to 100.0 |

### Example 22

| | % w/w |
|---|---|
| Toltrazuril | 2.0 |
| Emodepside | 0.1 |
| Praziquantel | 1.0 |
| Butylhydroxyanisole | 0.1 |
| Glyceryl dibehenate (Compritol 888ATO) | 5.0 |
| Sorbic acid | 0.08 |
| Sunflower oil | to 100.0 |

### Biological Examples

### A. Nematode Efficacy in Puppies

To determine the biological activity of the suspension against nematodes, puppies were infected artificially with infectious stages of various nematodes (see Table 1). After determining a patent infection, the animals were treated with the suspension at different dosage rates. With reference to studies on faeces, the reduction in egg elimination and the number of eliminated worms were determined. Five days after the treatment, a second treatment with a reference product was carried out. Thereafter, further studies of faeces were carried out, and the efficacies were calculated.

TABLE 1

| | | Emodepside | | |
|---|---|---|---|---|
| Number of | | dosage rate | Efficacy | |
| animals | Nematodes | [mg/kg BW] | [%] | Formulation |
| 6 per group | Uncinaria | 0.25 | 99.6 | Ex. 15 |
| | stenocephala | 0.1 | 85.7 | emodepside 0.1% |
| | | 0.05 | 67.3 | toltrazuril 2% |
| 6 per group | Uncinaria | 0.75 | 100 | Ex. 16 |
| | stenocephala | 0.5 | 100 | emodepside 0.1% |
| | | 0.25 | 100 | toltrazuril 2% |
| 6 per group | Toxocara | 0.05 | 50.5 | Ex. 17 |
| | canis | 0.125 | 98.7 | emodepside 0.1% |
| | | 0.25 | 100 | toltrazuril 4% |
| 8 per group | Ancylostoma | 0.25 | 94 | Ex. 18 |
| | caninum | 0.5 | 100 | emodepside 0.05% |
| | | | | toltrazuril 2% |
| 13 | Ancylostoma | 0.5 | 100 | Ex. 19 |
| | Caninum | | | emodepside 0.1% |
| | | | | toltrazuril 2% |

### B. Activity Against Isospora Species in Puppies

To determine the efficacy of the suspension against Isospora species in dogs, puppies were infected with infectious oocysts. After determining a patent infection on the basis of studies of faeces, the puppies were treated with different dosage rates, and the success of the therapeutic treatment was determined in comparison with an untreated control group. In naturally infected animals, too, the therapeutic efficacy of the suspension was determined. Furthermore, the success of a metaphylactic treatment was determined; here, puppies were treated 3 to 6 days post-infection, before patent oocyst elimination could be determined. The success of the treatment was determined on the basis of studies of faeces in comparison with an untreated control.

TABLE 2

| | | | Dosage rate of | | |
|---|---|---|---|---|---|
| Number of | | Treatment | toltrazuril | Efficacy | |
| animals | Isospora species | scheme | [mg/kg BW] | [%] | Formulation |
| 6 to 7 | Isospora | metaphylactic | 10 | >99 | Ex. 20 |
| per group | ohioensis | therapeutic | 10 | >99 | emodepside 0.1% |
| | comp. | | | | toltrazuril 2% |
| | | therapeutic | 20 | >99 | Ex. 21 |
| | | | | | emodepside 0.1% |
| | | | | | toltrazuril 4% |
| 7 | Isospora canis | therapeutic | 10 | >99 | Ex. 20 |
| | | | | | emodepside 0.1% |
| | | | | | toltrazuril 2% |
| 9 per | Isospora canis | metaphylactic | 10 | 99.8 | Ex. 10 |
| group | | therapeutic | 10 | 96.9 | emodepside 0.1% |
| | | | | | toltrazuri12% |

1. A preparation comprising a triazine which is active against parasitic protozoans and has a particle size d(90)≦15 μm and an anthelmintic cyclodepsipeptide in an oil base.

2. The preparation according to claim 1, where the triazine is a compound of the formulae (I) or (II) 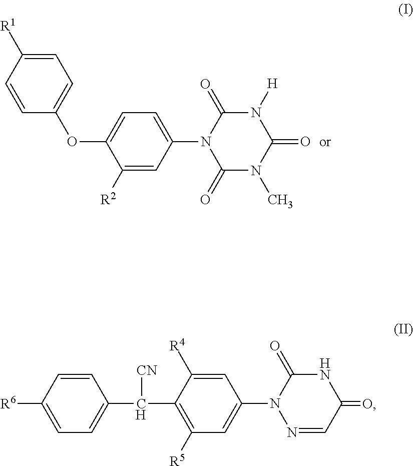
in which
R¹ represents R³—SO₂— or R³—S—,
R² represents alkyl, alkoxy, halogen or SO₂N(CH₃)₂ and
R³ represents haloalkyl,
R⁴ and R⁵ independently of one another represent hydrogen or Cl and
R⁶ represents fluorine or chlorine,
and their physiologically acceptable salts.

3. The preparation according to claim 2, where the triazine is toltrazuril, ponazuril or diclazuril.

4. The preparation according to claim 3, where the cyclic depsipeptide is a 24-membered octacyclodepsipeptide.

5. The preparation according to claim 4, where the cyclic depsipeptide is emodepside.

6. The preparation according to claim 4, where the cyclic depsipeptide is PF 1022.

7. The preparation according to claim 4, comprising a thickener, and wherein the thickener is a glycerol ester.

8. The preparation according to claim 7, wherein the glycerol ester is a glycerol diester with C12-C24 fatty acids.

9. The preparation according to claim 8, wherein the glycerol diester is glycerol dibehenate.

10. A preparation comprising:
(a) 2-4% by weight toltrazuril;
(b) 0.1-0.2% by weight emodepside;
(c) 2-5% by weight glyceryl dibehenate;
(d) 0.05-0.5% by weight butylhydroxytoluene;
(e) 0.02-0.1% by weight sorbic acid; and,
(f) to 100% by weight sunflower oil.

11. (canceled)

12. (canceled)

13. A method of controlling parasitic protozoans and endoparasites in animals comprising administering to an animal in need thereof an effective amount of the preparation of claim 10.
